(19) United States
(12) Patent Application Publication
(10) Pub. No.: US 2013/0330275 A1
(43) Pub. Date: Dec. 12, 2013

(54) MISFOLDED PROTEINS IN CANCER TREATMENT AND DIAGNOSIS

(71) Applicant: Neil Roy CASHMAN, Vancouver (CA)

(72) Inventor: Neil Roy CASHMAN, Vancouver (CA)

(21) Appl. No.: 13/766,005

(22) Filed: Feb. 13, 2013

Related U.S. Application Data
(63) Continuation of application No. 13/168,129, filed on Jun. 24, 2011, now abandoned.
Application No. 13/168,129 is a continuation of application No. 12/201,238, filed on Aug. 29, 2008, now abandoned.
(60) Provisional application No. 60/968,931, filed on Aug. 30, 2007.

(51) Int. Cl. A61K 51/10; A61K 39/00; C07K 16/30; A61K 39/395

(52) U.S. Cl. CPC A61K 51/1093; A61K 39/39558; A61K 39/0011; C07K 16/30
USPC 424/1.49; 435/7.23; 424/139.1; 424/185.1; 530/391.7; 530/391.3; 424/178.1

(57) ABSTRACT

Cancer cells are identified and inhibited using agents that bind to epitopes unique to misfolded forms of surface proteins presented by the cancer cells. In one embodiment, cancer cells are identified and treated using antibodies that bind to a YYX epitope available on the misfolded form of the prion protein, PrP, which has been identified on various cancer cell lineages.

### CROSS REFERENCE TO RELATED APPLICATION

This application is a continuation of U.S. Ser. No. 13/168,129 filed Jun. 24, 2011, which is a continuation of Ser. No. 12/201,238 filed Aug. 29, 2008, now abandoned, which claims priority to U.S. Provisional Application No. 60/968,931 filed Aug. 30, 2007, the disclosures of which are incorporated herein in their entirety by reference.

### FIELD OF THE INVENTION

This invention relates to misfolded proteins and their association with cancer cells. More particularly, the invention relates to agents that bind misfolded proteins and their use in detecting and managing cancer cells that present them.

### BACKGROUND TO THE INVENTION

About one-third of the population of the developed world is destined to die from cancer. Current treatment for cancers—including chemotherapy and radiotherapy—are based on killing cancer cells preferentially to normal cells, the so-called “therapeutic window” which accepts significant adverse effects for even marginal slowing of tumor growth. Specific treatments that spare normal cells are urgently needed.

An aspect of cancer biology that leads to specific vulnerability of tumor cells is the dysregulation of protein folding “quality control,” such that some proteins may expose abnormal surface to aqueous environments. Recognition of these abnormally exposed protein domains, designated Disease Specific Epitopes (DSE), will serve as a diagnostic cancer marker or cancer treatment target, and provide insight into abnormal cell growth in cancer.

A disease specific epitope for the prion protein (PrP) has recently been described as a diagnostic and treatment target for the transmissible spongiform encephalopathies (Paramithiotis et al, Nature Medicine 2003, 9(7):893). This prion DSE, defined by the tripeptide Tyr-Tyr-Arg, is exposed on the molecular surface of disease-misfolded PrPSc, but is buried in the antibody-inaccessible interior of the normal prion protein PrPC. The PrPC is abundantly expressed by normal circulating lymphoid and myeloid cells (Cashman et al, Cell 1990, 61(1):185), and plays a role in hematopoietic differentiation from CD34+ bone marrow stem cells (Dodelet and Cashman, Blood 1998, 91(5):1556). However, Tyr-Tyr-Arg surface immunoreactivity has never been detected on any normal cell, including splenocytes of mixed lineage, and dissociated brain cells (Paramithiotis Nature Med 2003).

### SUMMARY OF THE INVENTION

It has now been discovered that tumor cell lines display cell surface immunoreactivity for Tyr-Tyr-Arg. These tumor cell lines were from lymphoid (MOLT-4), myeloid (HL-60) and oligodendroglial lineages (MO3.13), which also possessed cell surface PrPC. Moreover, it was also found that deliberate denaturation of PrP null cells by brief exposure to low pH also evoked cell surface immunoreactivity for Tyr-Tyr-Arg. It is concluded that some tumor cells possess misfolded proteins at the cell surface, including but not limited to the prion protein, and that Tyr-Tyr-Arg binding agents can detect some misfolded proteins, including but not limited to PrPC.

The implications of the discovery of a cell surface cancer DSE are potentially great. The detection of Tyr-Tyr-Arg expressing cells in the peripheral blood or spinal fluid provides a diagnostic marker for leukemias, lymphomas, and other cancers. Moreover, the existence of a specific DSE for cancer cells provides for the development and use of vaccines or immunotherapies for multiple tumor types, including hematogenous and solid tumors of the brain and other organs.

Thus, in a general aspect, the present invention provides the use of agents that bind selectively to a misfolded surface protein presented by a cancer cell, in the treatment, detection or diagnosis of cancer, especially in a mammal including a human. Particularly useful agents are antibodies, which may kill cancer cells through many mechanisms, including complement-mediated lysis or antibody-dependent cell-mediated cytotoxicity (ADCC). Agents that are particularly useful in treatment are cytotoxins, including cytotoxic antibodies or antibody conjugates that bind selectively to the misfolded surface protein and are cytotoxic to the cancer cells presenting the misfolded surface protein.

In particular embodiments of the invention, the misfolded surface protein target is misfolded PrPC protein, or any other cancer cell surface protein that is misfolded and presents an epitope having a YYX motif.

Accordingly, in one aspect, the present invention provides a method for treating a subject to inhibit the growth or proliferation of a cancer cell presented by the subject, comprising the step of treating the subject with an effective amount of an agent that binds selectively to an epitope presented by a misfolded protein on the surface of a cancer cell. In embodiments, the epitope is YYX or an epitope that is unique to misfolded PrPC and is other than YYX (a “non-YYX PrP epitope”). Most suitably, the binding agent is, or comprises, a cytotoxic agent, or sensitizes the cancer cell to a cytotoxic agent. Suitably, the agent is an antibody that binds selectively to the epitope target, or a vaccine that induces formation of such antibody in a recipient. In embodiments, the binding agent is an anti-YYX antibody or a YYX binding fragment thereof, or a vaccine for production thereof. In related embodiments, the agent is a conjugate comprising a cytotoxin and the binding agent, such as an immunoconjugate. In other embodiments of the invention, the binding agent is administered in combination with one or more additional anti-cancer agents or treatment modalities.

In another of its aspects, the present invention provides a conjugate comprising a cytotoxin and an agent that selectively binds the target epitope. In embodiments, the conjugated cytotoxin is a chemotherapeutic agent, a toxin, and a radioisotope. In a related aspect, the present invention provides a pharmaceutical composition comprising the conjugate and a physiologically tolerable vehicle therefor.

Also provided, in another aspect of the invention, is a method for screening cancer cells, comprising the step of obtaining a sample comprising cancer cells, and assaying said cancer cells for the presence of the target epitope, i.e., the YYX epitope or a misfolded PrPC epitope other than YYX. In a related aspect, the screening is performed on a cancer cell sample obtained from a subject suspected of having cancer. In a related embodiment, a subject positive for the misfolded protein is treated using the method of the present invention. In embodiments, screening is performed using antibodies, and particularly labeled antibodies or binding fragments that bind the target epitope.

The present invention further provides, as an article of manufacture, a container comprising an agent that binds the target epitope and a label associated with the container, wherein the label indicates the contents of the container are useful in the treatment of a cancer.

These and other aspects of the present invention are now described in greater detail with reference to the accompanying Figures in which:

### BRIEF REFERENCE TO THE FIGURES

FIG. 1 shows MO313 cells stained with 4C2-FITC. Control (left) and PI-PLC treated (right); and

FIG. 2 shows MOLT4 cells stained with isotype control (left) and 4C2 antibody (right).

### DETAILED DESCRIPTION OF THE INVENTION

The invention relates to detection and treatment of cancer cells having a surface protein that is in a misfolded conformation and that, as a result of the misfolding, presents an epitope that is unique to the misfolded surface protein, relative to the normal conformation of that protein. Thus, an agent that binds “selectively” to the misfolded protein is an agent that binds to the misfolded form of the protein with greater affinity than to the normal conformation of the protein.

The binding agents useful in the present invention thus bind selectively to the misfolded protein, and preferably to the misfolded form of a human protein. In embodiments, this selective binding is achieved by targeting an epitope that is presented either only by the misfolded conformation of the protein, or in a more accessible form by the misfolded protein. The target protein can be any protein that is presented on the surface of a cancer cell in a misfolded conformation that can be recognized by an agent that binds selectively thereto. The agent can then be used to distinguish the misfolded surface protein from the protein in its normal conformation. These are the so-called disease specific epitopes or, more particularly, the cancer-specific epitopes.

In particular embodiments, the epitope target is an epitope that is presented by the cellular prion protein, PrPC, when in a misfolded state. In embodiments, this epitope is the YYX epitope. The “YYX” designation refers to the peptide sequence Tyr-Tyr-X, wherein X is any amino acid and is preferably Arg or, alternatively, is suitably Gln or Asp. Thus, the epitope that is targeted in accordance with embodiments of the present invention is YYD, YYQ or, preferably, YYR. The targeted epitope may comprise oxidized, nitrated or other derivatized or modified forms of the amino acids, either in isolation or in their peptide sequence context, that are formed endogenously by the microenvironmental conditions in which the surface protein is formed or presented. Examples of such derivatized forms of the amino acids include nitrotyrosine and, in the case of peptides, altered glycosylation.

As noted hereinabove, this YYX motif is present in the PrPC protein, but is not exposed when the PrPC protein is in its normal conformation. Through misfolding however, a conformational variant of the PrPC protein is formed and presented on the surface of cancer cells in which the YYX motif is exposed as a YYX epitope to which antibodies and other binding agents can be directed, in accordance with methods of the present invention.

It will be appreciated that the invention also applies to other epitopes that do not have a YYX motif yet are exposed, and can therefore be targeted, in conformational variants of the PrPC protein or, indeed, in any other cancer cell surface protein that undergoes misfolding.

In the case of misfolded PrPC, other epitopes that can usefully be targeted include the misfolded PrPC epitope recognized by the gene 5 protein (G5p; PDB ID code 1VQB), a single-stranded DNA-binding protein, and the DNA epitope recognized by the OCD4 antibody as described in more detail by Zou et al, in Proc. Natl. Acad. Sci., 2004, 101(5):1380, incorporated herein by reference. Other misfolded PrP epitopes also include those recognized by monoclonal antibodies with motif-grafted PrPSc-binding PrP peptides (Moroncini et al, Proc Natl Acad Sci USA. 2004 Jul. 13; 101(28):10404-9 and see WO03/085086 published Oct. 16, 2003 incorporated herein by reference), the monoclonal antibody 15B3 (Korth et al, Nature. 1997 Nov. 6; 390(6655):74-7, and see WO98/37210 published Aug. 27, 1998 incorporated herein by reference), and others (Curin-Serbec et al, J Biol Chem, 2004 Jan. 30; 279(5):3694-8 incorporated herein by reference). A hybridoma source of MAb 15B3 is on deposit under accession number DSM ACC2298.

In valuable embodiments of the present invention, agents that bind the target epitope, such as the YYX epitope, are exploited to detect and treat cancer cells. The term “cancer cells” is used herein with reference to cells, and tumours comprising such cells, that are characterized by unregulated cell growth. Cancer cells are thus characterized by neoplastic cell growth and proliferation, whether malignant or benign, and include all pre-cancerous and cancerous cells as well as tissues comprising such cells. The term “cancer cells” includes human cancer cells and cancer cells from other mammals including pets, and livestock including horses.

The present treatment method results in the inhibition of “growth or proliferation” of cancer cells presenting a YYX epitope. At the in vitro level, inhibition of such growth or proliferation is revealed by a reduction in the number, size, viability, growth rate, proliferation rate, or metabolic activity of the cancer cells that are treated, relative to an untreated control sample. At the in vivo level, such inhibition of growth or proliferation can further be revealed as a reduction in the growth rate, size, number or metastatic status of tumours harbouring cancer cells that present the target epitope. It will be appreciated that all of these end-points can readily be determined using assays and procedures that are well established in the oncology field for this purpose, and with the aid of agents that detect the target epitope, as provided by the present invention and as detailed further herein.

YYX Binding Agents

In embodiments, the present invention makes use of agents that bind the YYX epitope, and preferably agents that bind the YYR epitope. It will be appreciated that YYX binding agents can be provided in a variety of different forms, including antibodies and binding fragments thereof, peptides and proteins, aptamers including DNA and peptide aptamers, retroenantiomeric forms of YYX, and small molecules (generally smaller than 1,000 daltons in size, and preferably less than about 500 daltons in size).

In preferred embodiments, the YYX binding agent binds selectively to the YYX motif. Selective-binding agents are agents that bind the YYX motif, and bind proteins that present the motif in a solvent-accessible orientation, with an affinity that is at least one order of magnitude greater (e.g., at least 2, 3, 4 or 5 orders of magnitude greater) than the affinity with which they bind a different, unrelated motif. For instance, the binding affinity of the YYX binding agent is preferably at least an order of magnitude greater than its binding affinity for the PrPC protein. Relative binding affinities can be determined, and the YYX binding agent so selected, on the basis of assays and techniques that generally are well established in the art for this purpose. In embodiments of the invention, the YYX binding agent is an agent that binds YYD, YYQ or YYR. In a preferred embodiment the YYX binding agent is an agent that binds YYR. To be most effective in inhibiting the growth or proliferation of cancer cells that present the YYX epitope, the YYX binding agent desirably is a cytotoxic agent or is conjugated to a cytotoxic agent. In the alternative, the YYX binding agent has the property of sensitizing the cancer cell to the effects of a cytotoxin or a given treatment modality, such as radiation. Also alternatively, the binding agent can exert its anti-cancer activity by ligating the target protein to prevent signaling therefrom through its interaction with neighbouring proteins, or by stabilizing the target protein to prevent or preserve signaling or other events that are driven by its conformational change.

YYX Antibodies

In a preferred embodiment, the YYX binding agent is an anti-YYX antibody or a YYX binding fragment thereof. In a particularly preferred embodiment, the anti-YYX antibody or binding fragment is a YYR antibody or a YYR binding fragment thereof. As noted above, the YYX or YYR antibody or binding fragment thereof desirably binds selectively to the YYX epitope.

In preferred embodiments, the YYX antibody is a high affinity antibody, having an affinity constant for binding to PrPSc that is less than 10 uM, preferably less than 1 uM, more preferably less than 100 nM and most preferably less than 10 nM.

The production of anti-YYX-antibodies and particularly anti-YYR antibodies has been described in the prior art, and particularly in U.S. Pat. No. 7,041,807, Cashman et al published May 9, 2006, the entire disclosure of which is incorporated herein by reference in its entirety. Specific protocols useful in the production of YYR antibodies are reproduced in the examples section herein. More generally, it will be appreciated that antibodies useful in the present invention include the various intact forms including polyclonal antibodies, monoclonal antibodies, and recombinant antibodies including chimeric antibodies, humanized antibodies as well as fully human antibodies. The chimeric antibodies comprise a portion of the heavy and/or light chain that is homologous with corresponding sequences in antibodies derived from a particular species, or belonging to a particular antibody class or subclass, while the remainder of the chain(s) is homologous with corresponding sequences derived from another species or belonging to a different antibody class. Humanized antibodies are chimeric antibodies that comprise minimal sequence derived from non-human antibody, usually incorporating CDRs from a non-human antibody into a human antibody framework, which may further be altered to incorporate non-human residues that restore and enhance antigen binding. The “fully” human antibodies can be produced in a non-human host using various techniques that are now established, including through the use of phage display libraries, and particularly by introducing human immunoglobulin loci into transgenic animals such as mice in which the endogenous immunoglobulin genes have been partially or completely inactivated. Upon challenge, human antibodies are produced which closely resemble that seen in humans in most respects, including gene rearrangement, assembly and antibody repertoire. The antibodies may be of any useful class, including IgA, IgD, IgE, IgG and IgM, and isotypes including IgG1, IgG2, IgG3, and IgG4.

To permit their use as cytotoxins per se, to inhibit directly the growth or proliferation of cancer cells presenting the YYX epitope, the antibodies can exert their anti-cancer activity through endogenous mechanisms such as complement-mediated cytotoxicity (CDC) and/or antibody-dependent cellular cytotoxicity (ADCC). It will be appreciated that the antibodies can be engineered or selected to have altered effector function, to enhance effectiveness in treating cancer. Cysteine residues, for instance, may be introduced to the Fc region to allow interchain disulfide bond formation. The resulting homodimeric antibody may have improved internalization capacity, and more importantly may have increased complement dependent cytotoxicity (CDC) and/or ADCC activities. Homodimeric antibodies with enhanced anti-tumour activity may also be prepared using heterobifunctional cross-linkers as described in Wolff et al, Cancer Research 53:2560-2565 (1993). Alternatively, and antibody can be engineered which has dual Fc regions and enhanced CDC and ADCC activity.

Antibody fragments useful in the present invention include the YYX binding fragments of anti-YYX antibodies, including Fab, Fab′, F(ab′)2, and Fv fragments, diabodies, linear antibodies, single chain antibody molecules, and multispecific antibodies formed from antibody fragments. Antibody fragments that incorporate the Fc region can also be engineered or conjugated as noted above to provide altered effector function, thereby to enhance ADCC and/or CDC activity.

It will further be appreciated that the YYX antibody can be substituted by any other agent that binds with selectivity to the YYX epitope, including small molecules, peptides, aptamers and the like. Moreover, the YYX antibody can be substituted by any agent that binds selectively to an epitope other than YYX that is unique to misfolded PrPC, or binds to any epitope presented uniquely by the misfolded from of any cancer cell surface protein. Thus, in an embodiment, the binding agent is the gene 5 protein (G5p) which binds selectively to misfolded PrPC, or any fragment thereof that binds selectively to misfolded PrPC. In another embodiment, the binding agent is the MAb 15B3 deposited under accession number DSM ACC2298, or a fragment thereof that binds selectively to misfolded PrPC. In another embodiment, the binding agent is the OCD4 antibody or a fragment thereof that binds selectively to misfolded PrPC. In a further embodiment, the binding agent is a MAb comprising a grafted motif that binds selectively to misfolded PrPC as described for instance by Moroncini et al, supra.

Vaccines

Binding agents that are antibodies can be provided in vaccine form comprising an immunogen that elicits antibody to the target epitope in the vaccine recipient. Thus, according to one embodiment of the present invention, the treatment of a subject to introduce antibodies can be achieved by administering to the subject a vaccine that elicits production of the antibodies by the subject, such as a vaccine that elicits endogenous YYX antibody. Such vaccines comprise an immunogen and, suitably, an adjuvant or other suitable vaccine carrier. The immunogen can be in the form of a peptide that presents the YYX epitope in a context recognized as foreign by the subject, to raise an immune response thereto and thereby foster the production of endogenous YYX antibody. Suitably, the immunogen comprises the peptide and a conjugated carrier molecule. Immunogenicity of the peptide can be further enhanced by addition of the adjuvant or carrier, in accordance with standard practice. To raise endogenous antibody, routes of administration, antigen doses, number and frequency of injections will vary from species to species and may parallel those currently being used in the clinic and/or experimentally to provide immunity or therapy against cancer. For example, the vaccines are pharmaceutically acceptable compositions containing the YYX-presenting peptides, its analogues or mixtures or combinations thereof, in an amount effective in the mammal, including a human, treated with that composition to raise immunity sufficient to protect the treated mammal from prion infection for a period of time.

Different types of vaccines can be developed according to standard procedures known in the art. For example, a vaccine may be peptide-based, nucleic acid-based, bacterial or viral-based vaccines. More specifically, with regard to peptide vaccines, peptides corresponding to the YYX epitope or a functional derivatives thereof can be utilized as a prophylactic or therapeutic vaccine in a number of ways, including: 1) as monomers or multimers of the same sequence, 2) combined contiguously or non-contiguously with additional sequences that may facilitate aggregation, promote presentation or processing of the epitope (e.g., class I/II targeting sequences) and/or additional antibody, T helper or CTL epitopes to increase the immunogenicity of the PrPSc specific epitope as a means to enhance efficacy of the vaccine, 3) chemically modified or conjugated to agents that would increase the immunogenicity or delivery of the vaccine (e.g., fatty acid or acyl chains, KLH tetanus toxoid, cholera toxin, etc.) 4) any combination of the above, 5) the above in combination with adjuvants, including but not limited to aluminum salts, saponins or triterpenes, MPL, and cholera toxin, and/or delivery vehicles, including but not limited to liposomes, VPLs or virus-like particles, microemulsions, attenuated or killed bacterial and viral vectors, and degradable microspheres, 6) administered by any route or as a means to load cells with antigen ex vivo.

Examples of uses of nucleic-acid based vaccines as a prophylactic or a therapeutic include: 1) any nucleic acid encoding the expression (transcription and/or translation) of a peptide presenting the YYX epitope, 2) additional nucleic acid sequences that facilitate processing and presentation, aggregation, secretion, targeting (to a particular cell type) of the epitope, either translational fusions or independent transcriptional units, 3) additional nucleic acid sequences that function ad adjuvants/immunomodulators, either translational fusions or independent transcriptional units, 4) additional antibody, T helper or CTL epitopes that increase the immunogenicity of the epitope or efficacy of the vaccine, either translational fusions or independent, 5) any combination of the above, 6) the above administered in saline (‘naked’ DNA) or in combination with a adjuvant(s), (e.g. aluminum salts, QS-21, MPL), immunomodulatory agent(s) (e.g. riIL-2, rGM-CSF,rIL-12), and/or nucleic acid delivery agents (e.g. polymer-, lipid-, peptide-based, degradable particles, microemulsions, VPLs, attenuated bacterial or viral vectors) using any route or ex vivo loading.

Attenuated or killed bacterial or viral vectors can be used to deliver either the antigen or DNA/RNA that codes for the expression of the antigen. These can also be used as a means to load cells with antigen ex vivo.

Peptides suitable for use in vaccines, and to raise antibodies that bind the YYX epitope for direct use in subjects, are peptides that incorporate the YYX sequence, and include peptides such as those of the formula:

(SEQ ID NO: 1)
A-Tyr-Tyr-B-(Tyr-Tyr-B)n,
or
(SEQ ID NO: 2)
A-Tyr-Tyr-B-C-Tyr-Tyr-D-Tyr-Tyr-(Tyr-Tyr-B)n

wherein A, B, C and D are, independently, any amino acid or absent; and n is any integer from 0 to 10.

Preferably at least one of A, B, C and D is not Tyr. In other embodiments, A, B, C or D are chosen independently from Ala, Cys, Asp, Glu, Phe, Gly, H is, Ile, Lys, Leu, Met, Asn, Pro, Gln, Arg, Ser, Thr, Val or Trp. In still other preferred embodiments, A is chosen from Ala, Cys, Asp, Glu, Phe, Gly, His, Ile, Lys, Leu, Met, Asn, Pro, Gln, Arg, Ser, Thr, Val or Trp and B, C and D are chosen independently from Arg, Gln, Asp, Glu, Phe or Trp. An exemplary peptide is A-Tyr-Tyr-Arg-Arg-Tyr-Tyr-Arg-Tyr-Tyr (SEQ ID NO: 3) or a pharmaceutically acceptable salt thereof, preferably linked to an immunological carrier such as KLH, via a bridging Cys residue.

For active immunization using vaccines comprising YYX peptides, the dose optionally ranges from about 0.0001 microgram to 10 grams, about 0.01 microgram to about 1 gram, about 1 microgram to about 1 mg, and about 100 to 250 micrograms per treatment. In one embodiment the timing of administering treatment is at one or more of the following: 0 months, 2 months, 6 months, 9 months, and/or 12 months. In one regimen, the dosing is at 2, 6, 9, and 12 months following the first immunization. In another regimen, the dosing is at 2 and 4 weeks following the first immunization, and then monthly afterwards. In an alternative regimen, the dosing varies depending on the physiological condition of the subject and/or the response to the subject to prior immunizations. The route of administration optionally includes, but is not limited to, intramuscular and intraperitoneal injections. In one embodiment the composition is injected into the deltoid muscle.

Conjugates

For use in the present method, and according to an aspect of the present invention, there is provided a conjugate that comprises an agent that binds target epitope and a cytotoxin. In embodiments, the conjugate comprises a cytotoxin and an agent that binds selectively to the YYX epitope. “Cytotoxin” refers to a compound including a chemotherapeutic or a radiotherapeutic compound and the like that is useful therapeutically to reduce the viability of cancer cells, e.g., to inhibit the growth and/or proliferation of the cancer cells.

The YYX binding agent and the cytotoxin may be conjugated through non-covalent interaction, but more desirably, are coupled by covalent linkage either directly or, more preferably, through a suitable linker. In a preferred embodiment, the conjugate comprises a cytotoxin and a YYX antibody, to form an immunoconjugate. Immunoconjugates of the antibody and cytotoxin are made using a variety of bifunctional protein coupling agents such as N-succinimidyl-3-(2-pyridyldithiol) propionate, iminothiolane, bifunctional derivatives of imidoesters such as dimethyl adipimidate HCL, active esters such as disuccinimidyl suberate, aldehydes such as glutaraldehyde, bis-azido compounds such as bis-(p-diazoniumbenzoyl)-ethylenediamine), diisocyanates such as tolyene 2,6-diisocyanate, and bis-active fluorine compounds (such as 1,5-difluoro-2,4-dinitrobenzene). Carbon-14-labeled 1-isothiocyanobenzyl-3-methyldiethylene triaminepentaacetic acid (MX-DTPA) is a chelating agent suitable for conjugation of radio nucleotide to the antibody.

The cytotoxin component of the immunoconjugate can be a chemotherapeutic agent, a toxin such as an enzymatically active toxin of bacterial, fungal, plant or animal origin, or fragments thereof, or a small molecule toxin), or a radioactive isotope such as ²¹²Bi, ¹³¹I, ¹³¹In, ⁹⁰Y, and ¹⁸⁶Re, or any other agent that acts to inhibit the growth or proliferation of a cancer cell.

Chemotherapeutic agents useful in the generation of such immunoconjugates include adriamycin, doxorubicin, epirubicin, 5-fluoroouracil, cytosine arabinoside (“Ara-C”), cyclophosphamide, thiotepa, busulfan, cytoxin, taxoids, e.g. paclitaxel, and docetaxel, toxotere, methotraxate, cisplatin, melphalan, vinblastine, bleomycin, etoposide, ifosgamide, mitomycin C, mitoxantrone, vincristine, vinorelbine, carboplatin, teniposide, daunomycin, caminomycin, aminopterin, dactinomycin, mitomycins, esperamicins, 5-FU, 6-thioguanine, 6-mercaptopurine, actinomycin D, VP-16, chlorambucil, melphalan, and other related nitrogen mustards. Also included are hormonal agents that act to regulate or inhibit hormone action on tumors such as tamoxifen and onapristone.

Toxins and fragments thereof which can be used include diphtheria A chain, nonbonding active fragments of diphtheria toxin, cholera toxin, botulinus toxin, exotoxin A chain (from Pseudomonas aeruginosa), ricin A chain, abrin A chain, modeccin A chain, alpha-sarcin, Aleurites fordii proteins, dianthin proteins, phytolaca Americana proteins (PAPI, PAPII, and PAP-S), momordica charantia inhibitor, curcin, crotin, sapaonaria, officinalis inhibitor, gelonin, saporin, mitogellin, restrictocin, phenomycin, enomycin, and the tricothcenes. Small molecule toxins include, for example, calicheamicins, maytansinoids, palytoxin and CC1065.

It will further be appreciated that the antibody component of the conjugate can be substituted by any other agent that binds with selectivity to the YYX epitope, including peptide mimetics including retro-inverso equivalents, small molecules, peptides, aptamers and the like. Moreover, the antibody component of the conjugate can be substituted by any agent that binds selectively to a PrPC epitope other than YYX, or binds to any epitope presented uniquely by the misfolded from of a cancer cell surface protein. Thus, in an embodiment, the conjugate comprises a cytotoxin and the gene 5 protein (G5p), which binds misfolded PrPC. In another embodiment, the binding agent is the MAb 15B3 deposited under accession number DSM ACC2298, or a fragment thereof that binds selectively to misfolded PrPC. In another embodiment, the binding agent is the OCD4 antibody or a fragment thereof that binds selectively to misfolded PrPC. In a further embodiment, the binding agent is a peptide derived from PrP that binds selectively to misfolded PrPC as described for instance by Moroncini et al, supra.

Pharmaceutical Compositions

Therapeutic formulations of the antibody or the conjugate are prepared for storage by mixing the antibody or conjugate having the desired degree of purity with optional pharmaceutically acceptable carriers, excipients or stabilizers (Remington's Pharmaceutical Sciences, 16^{th} edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl, or benzyl alcohol; alkyl parabens such as methyl or propyl paraben; catechol; resorcinol; cyclohexanol; 3-pentanol; and m-cresol); low molecular weight (less than about 10 residues) polypeptides; proteins such as serum, albumin, gelatin, or immunoglobulins; hydrophilic polymers such as polyvinylpyrrolidone; amino acids such as glycine, glutamine, asparagines, histidine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, PLURONICS or polyethylene glycol (PEG).

Non-antibody compounds that bind YYX epitope can be formulated in an analogous manner, using standard techniques well known in the art.

The active ingredients to be used for in vivo administration must be sterile. This is readily accomplished by filtration through sterile filtration membranes.

Sustained-release preparations may be prepared. Suitable examples of sustained-release include semipermeable matrices of solid hydrophobic polymers containing the antibody, which matrices are in the form of shapes articles, e.g., films or microcapsules. Examples of sustained-release matrices include polyesters, hydrogels (for example, poly (2-hydroxyethyl-methacrylate), polyactides (U.S. Pat. No. 3,773,919), copolymers of L-glutamic acid and ethyl-L-glutamate, non-degradable ethylene-vinyl acetate, degradable lactic acid-glycolic acid copolymers such as injectable microspheres composed of lactic acid-glycolic acid copolymer and leuprolide acetate, and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Pharmaceutical Combinations

Administration “in combination with” one or more further therapeutic agents includes simultaneous (concurrent) and consecutive administration in any order.

Other therapeutic regimens may be combined with the administration of the anti-cancer agents, e.g., antibodies or conjugates, of the instant invention. For example, the patient to be treated with such anti-cancer agents may also receive radiation therapy, such as external beam radiation. Alternatively, or in addition, a chemotherapeutic agent may be administered to the patient. Preparation and dosing schedules for such chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992). The chemotherapeutic agent may precede, or follow administration or the anti-tumor agent, e.g., antibody, or may be given simultaneously therewith. The antibody may be combined with an anti-estrogen compound such as tamoxifen or an anti-progesterone such as onapristone (see, EP 616812) in dosages known for such molecules.

It may be desirable to also administer antibodies or conjugates against other tumor associated antigens, such as antibodies which bind to the ErbB2, EGFR, ErbB3, ErbB4, or vascular endothelial factor (VEGF). Alternatively, or in addition, two or more antibodies binding that same or two or more different antigens disclosed herein may be co-administered to the patient. Sometimes it may be beneficial to also administer one or more cytokines to the patient. In a preferred embodiment, the antibodies herein are co-administered with a growth inhibitory agent. For example, the growth inhibitory agent may be administered first, followed by an antibody of the present invention. However, simultaneous administration or administration of the antibody of the present invention first is also contemplated. Suitable dosages for the growth inhibitory agent are those presently used and may be lowered due to combined action (synergy) of the growth inhibitory agent and the antibody herein. Further, the YYX antibody or fragment may be administered in combination with a vaccine for raising YYX antibody, providing both active and passive immunotherapy to the recipient.

Kits

In another embodiment of the invention, an article of manufacture containing materials useful for the diagnosis or treatment of the disorders described herein is provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or vial having a stopper pierceable by a hypodermic injection needle). The label on, or associated with, the container indicates that the composition is used for treating a cancer condition. The article of manufacture may further compromise a second container compromising a pharmaceutically-acceptable buffer, such as phosphate-buffered saline, Ringer's solution and dextrose solution. It may further include other matters desirable from a commercial and use standpoint, including other buffers, diluents, filters, needles, syringes, and package inserts with instructions for use.

Dosing and Administration

An anti-cancer therapeutic according to the invention may be administered with a pharmaceutically-acceptable diluent, carrier, or excipient, in unit dosage form.

Any appropriate route of administration can be employed, for example, parenteral, intravenous, subcutaneous, intramuscular, intracranial, intraorbital, ophthalmic, intraventricular, intracapsular, intraspinal, intracisternal, intraperitoneal, intranasal, aerosol, or oral administration.

For the treatment of subjects presenting with cancer cells having the YYX epitope, the appropriate dosage of an anti-tumor agent, e.g., an antibody, fragment or conjugate, will depend on the type of disease to be treated, as defined above, the severity and course of the disease, whether the agent is administered for preventative or therapeutic purposes, previous therapy, the patients clinical history and response to the agent, and the discretion of the attending physician. The agent is suitably administered to the patient at one time or over a serious of treatments.

For example, depending on the type and severity of the disease, about 1 μg/kg to 15 mg/kg (e.g., 0.1-20 mg/kg) of antibody or conjugate is a candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

The YYX binding agents are useful in the treatment of a variety of cancers, to inhibit the growth or proliferation of cancer cells and tumours comprising them, including hematopoietic cell cancers and solid tumours. Exemplary conditions or disorders to be treated with YYX binding agents include benign or malignant tumors (e.g., renal, liver, kidney, bladder, breast, gastric, ovarian, colorectal, prostate, pancreatic, lung, vulva, and thyroid); hepatic carcinomas; sarcomas; glioblastomas; and various head and neck tumors; leukemias and lymphoid malignancies. In particular embodiments, the YYX binding agents are used in the treatment of such cancer cells that express YYX epitope, as determined by the screening assays herein described. In particular embodiments, the cancer cells are YYX-presenting cancer cells of the myeloid, lymphoid or oligodendroglial lineages.

It will be appreciated that subjects who could benefit from the present method include mammals including humans as well as livestock, and pets.

Screening for Equivalent Agents

Novel YYX-binding agents useful in the present method may be identified using the antibodies of the invention. For example, combinatorial chemical libraries or small molecule libraries are screened to identify compounds having the ability to inhibit the binding interaction of one or more anti-YYX antibodies to a YYX epitope according to standard methods (e.g., equilibrium dialysis, Biacore analysis, or competitive inhibition). Such libraries may be derived from natural products, synthetic (or semi-synthetic) extracts, or chemical libraries according to methods known in the art. Those skilled in the field of drug discovery and development will understand that the precise source of compounds is not critical to the screening procedure(s) of the invention. Examples of natural compound sources include, but are not limited to plant, fungal, prokaryotic, or animal sources, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries may be obtained commercially or may be produced according to methods known in the art. Furthermore, if desired, any library or compound is readily modified using standard chemical, physical, or biochemical methods.

As well, the invention features a method for identifying a compound for the treatment of cancer, wherein the method includes the steps of (a) measuring the binding of an anti-YYX antibody to misfolded PrPC (or PrPSc) in the presence of a test compound; wherein a level of binding of the anti-YYX antibody to PrPC (or PrPSc) in the presence of the test compound that is less than the level of binding of the anti-YYX antibody to misfolded PrPC (or PrPSc) in the absence of the test compound is a indication that the test compound is a potential therapeutic compound for the treatment of a cancer. Preferably, the anti-YYX antibody is an anti-YYR antibody, anti-YYD antibody, or anti-YYQ antibody.

Screening for YYX Positive Cancer Cells

Antibodies and fragments thereof that bind selectively to the target epitope, e.g. the YYX epitope, are used, in accordance with an aspect of the invention, to screen cancer cells to detect those which present the YYX epitope. In a preferred embodiment, screening is applied to a sample of cancer cells taken from a subject that is a candidate for YYX antibody therapy. Subjects testing positive for cancer cells that present the YYX epitope can then be scheduled for therapy with an agent that binds the YYX epitope, in accordance with the method of the present invention.

Standard techniques, combined with the antibodies or other binding agents herein described can be used to screen cancer cells. Desirably, the antibodies incorporate a detectable label. The label may be detectable by itself. (e.g., radio-isotope labels or fluorescent labels) or, in the case of an enzymatic label, may catalyze chemical alteration of a substrate compound or composition which is detectable. Radionuclides that can serve as detectable labels include, for example, I-131, I-123, I-125, Y-90, Re-188, Re-186, At-211, Cu-67, Bi-212, AND Pd-109.

In situ detection of the binding to cancer cells bearing a target epitope can be performed, using YYX antibodies for example, by immunofluorescence or immunoelectron microscopy. For this purpose, a histological specimen is removed from the patient, and a labeled antibody is applied to it, preferably by overlaying the antibody on a biological sample. This procedure also allows for distribution of the YYX epitope to be examined within biopsied tumour tissue. It will be apparent for those skilled in the art that a wide variety of histological methods are readily available for in situ detection.

More particularly, YYX antibodies or binding fragments or other YYX binding agents may be used to monitor the presence or absence of YYX reactivity in a biological sample (e.g., a tissue biopsy, a cell, or fluid) using standard detection assays. Immunological assays may involve direct detection, and are particularly suited for screening large amounts of samples for the presence of YYX-presenting cancer cells. For example, polyclonal or monoclonal antibodies produced against a continuous YYX epitope such as YYR (as described above) may be used in any standard immunoassay format (e.g., ELISA, Western blot, immunoprecipitation, flow cytometry or RIA assay) to measure complex formation. Any appropriate label which may be directly or indirectly visualized may be utilized in these detection assays including, without limitation, any radioactive, fluorescent, chromogenic (e.g., alkaline phosphatase or horseradish peroxidase), or chemiluminescent label, or hapten (for example, digoxigenin or biotin) which may be visualized using a labeled, hapten-specific antibody or other binding partner (e.g., avidin). Exemplary immunoassays are described, e.g., in Ausubel et al., supra, Harlow and Lane, Antibodies: A Laboratory Approach, Cold Spring Harbor Laboratory, New York (1988), and Moynagh and Schimmel, Nature 400:105, 1999. For example, using the antibodies described herein, YYX epitope is readily detected at the cell surface using standard flow cytometry methods. Samples found to contain increased levels of labeled complex compared to appropriate control samples are taken as indicating the presence of YYX epitope, and are thus indicative of a cancer and amenable to treatment with the present antibodies.

### EXAMPLES

The production and evaluation of YYX antibody described below is reproduced from U.S. Pat. No. 7,041,807, for reference. In this example, reference is made to PrPSc. This scrapie form of the PrP protein is an aggregated form of PrPC that comprises misfolded PrPC and presents the YYR epitope, to which that present invention is directed in certain embodiments. Thus, antibodies raised against YYR bind this epitope when it is presented in a misfolded form, in this case within the context of the PrP aggregate known as PrPSc.

Polyclonal antisera, pAbC2, were raised in rabbits against a YYR peptide linked to KLH. Serum was collected from each rabbit after the immunization regime and total lgG purified using a Protein A column. The samples were then tested in immunoprecipitation reactions using brain homogenates from normal or scrapie-infected mice. Initial analysis of the brain homogenates used in these studies revealed detectable amounts of PrPC in normal brain extracts and PrP in infected samples. Incubation of these brain homogenates with BSA-coupled magnetic beads failed to precipitate any detectable PrP whereas, incubation of the samples with beads coupled with 6H4 (a PrP-specific monoclonal antibody) immunoprecipitated PrPC from normal brains, and PrPSc and PrP 27-30 from infected brains. When pAbC2 lgG was coupled to the beads and incubated with normal brain homogenates, no detectable PrP was immunoprecipitated. Strikingly, incubation of the pAbC2 lgG-coupled beads with infected samples immunoprecipitated PrPSc and PrP 27-30. Once again, these tissues harbored detectable amounts of PrPC and PrPSc and BSA-coupled beads failed to immunoprecipitate any PrP.

In addition, similar experiments showed that pAbC2 lgG specifically immunoprecipitates bovine PrPSc from BSE infected brains compared to 6H4. Moreover, pAbC2 antibodies can recognize bovine PrPSc in an ELISA system using soluble PC2 (prion receptor) as a capture reagent, but generates no signal in ELISA studies in which recombinant bovine PrPC is directly adsorbed to plates, despite its detectability using the 6H4 monoclonal antibody. Anti-YYR lgG does not recognize denatured recombinant bovine PrPC in western blotting (data not shown), similar to studies detailed below with mouse anti-YYR monoclonal antibodies.

Goat polyclonal antisera were also raised to YYR linked to KLH. Serum was collected and total lgG was isolated by ammonium sulfate precipitation. YYR-reactive lgG was also purified from the same sera by affinity chromatography using a YYR-conjugated column. These antisera were subjected to a similar set of screening and validating immunoprecipitation reactions as detailed above. One of the three immunized goats developed antiserum that was specific for PrPSc and was further characterized. As in previous experiments, 6H4-coupled beads non-discriminately precipitated both PrPC and PrPSc from infected mouse brains. No 6H4 immunoprecipitation of PrP 27-30 was seen in this experiment as is occasionally observed for unknown reasons. When total lgG from immunized goats was coupled to the beads, little, if any, material was precipitated from normal and infected mouse brain homogenates. In contrast, when YYR-affinity purified lgG was coupled to the magnetic beads, only PrPSc was precipitated from infected mouse brains.

In addition to the goat polyclonal antibody generation, monoclonal antibodies against the same PrPSc specific epitope were also generated, but with a derivative of the original antigen in which multiples of the original YYR peptide were linked together into one contiguous sequence. YYRRYYRYY (SEQ ID NO: 4) was synthesized in the attempt to increase the number of YYR epitopes in the peptide sequence, and to increase the chance of tyrosine stacking and/or frequency of pi-stacking. Moreover, one of the YYR sequences in the prion protein is preceded by an arginine in the five species of interest. The YYRRYYRYY (SEQ ID NO: 4) peptide was linked to KLH and mice were subsequently immunized with the antigen. Splenocytes from these mice were isolated and fused to the FO murine B cell line (ATCC CRL-1646) to generate specific hybridoma clones. Ascites were produced from clones that reacted with YYR conjugated to an alternative carrier, 8map, in an ELISA. lgG from these ascities were purified using a Protein-A column and screened and validated using standard methods. Five monoclonal antibodies were identified that specifically recognized PrPSc in immunoprecipitation reactions using brain homogenates from infected mice.

Continued evaluation of the PrPSc specific monoclonal antibodies revealed that they were capable of recognizing different murine strains of PrPSc through a conformationally dependent epitope. Antibody 1A4 was used in immunoprecipitations on numerous extracts prepared from different mice infected with either the MeI or 139A strain of murine scrapie. In these experiments, 1A4 was found to specifically precipitate PrPSc and PrP 27-30, regardless of the strain. This was also found for the other PrPSc specific monoclonal antibodies. When the brain homogenates (normal, ME7 and 139A infected) were electrophoresed in an SDS-PAGE gel under non-reducing conditions and then probed for PrP with one of the PrPSc specific monoclonal antibodies (1A4 or 6B1) or 5H4, it was clearly evident that only 6H4 was capable of detecting denatured PrPC and PrPSc. For 1A4 and 6B1, the PrPSc specific determinants had been lost upon denaturation of the sample, establishing the conformational sensitivity of the epitope.

In addition, it was determined that YYR-reactive monoclonal antibodies do not recognize any cell surface proteins on the surface of splenocytes or dissociated immediately ex-vivo brain cells from normal and PrP−/− knockout mice. Viable mouse splenocytes and brain cells were isolated by centrifugation of spleen cell and brain suspensions through a ficol gradient. Splenocytes were stained with the FITC-conjugated antibodies listed (solid lines), or with isotype-matched FITC labeled control antibodies. Non-viable cells were excluded from the analysis with propidium iodide. The lack of surface immunoreactivity on splenocytes or brain cells indicates that the YYR conformational epitope is rare, as suggested by the structural searches noted above. Moreover, the lack of appreciable signal provides an acceptable background for studies of PrPSc immunoreactivity at the cell surface of splenocytes and other test cells. Detection of cell surface PrPSc is useful as a diagnostic test for human animal prion disease infection. Finally, the lack of cell surface immunoreactivity provided an independent verification of the fact that anti-YYR antibodies do not recognize normal PrPC, as splenocytes and brain cells possess detectable surface PrPC by 6H4 immunohistochemistry.

Preparation of the Immunizing YYR Peptides

In order to develop an antibody to the YYR epitope presented by PrPSc and by misfolded PrPC but not by PrPC in its normal conformation, a peptide with the amino acid sequence (SEQ ID NO:5) Acetyl-Cys-Tyr-Tyr-Arg-NH2 (YYR) was synthesized, conjugated to KLH, and injected intramuscularly into rabbits using well known techniques.

At the amino-terminus of the peptide, a cysteine residue was added to allow conjugation of the peptide with the protein carrier. The amino group of the peptide was blocked by acetylation, and the carboxylic group of the peptide was blocked by amidation.

Peptide Synthesis

Peptides were synthesized using solid phase peptide synthesis methods either manually or automated (MPS396 peptides synthesizer, Advanced ChemTech). Coupling of amino acid residues was accomplished using Fmoc peptide synthesis chemistry (Fields et al., 1990, IJPPR 35, 161). Syntheses were performed on Wang or on amide Rink resins, with full side chain protection of amino acids. Since the alpha-NH2 groups of the amino acids were protected with the Fmoc group, the following protective groups were chosen for the side groups of the trifunctional amino acids:

Cysteine: 5-triphenylmethyl (Trt)
Arginine: 2,2,4,6,7-pentamethyldihydrobenzofuran-5 sulfonyl (Pbf)
Tyrosine: tert.-butyl ether (tBu)\
BOP, PyBOP, or TBTU were used as activation agents, depending on the chemistry and difficulty of the coupling reaction. All chemicals were purchased from Advanced Chem Tech, Bachem, and Calbiochem/NovaBiochem. Formation of each peptide bond between residues of the sequence was ensured by using a 3 to 6 fold excess of coupling reagents and by so-called double coupling; meaning that the coupling reaction was repeated for each amino acid added to the growing peptide chain.

Cleavage of Fluo-Peptides from Resin

After synthesis, the peptides were cleaved from the resin using the Reagent K as a cleavage mixture: water (2.5%), TIS (2.5%), EDT (2.5%), TFA (92.5%). The peptides were then precipitated with cold diethyl ether. The precipitates were centrifuged, washed three times with diethyl ether, dissolved in 20%-50% AcCN/water mixture, and lyophilized. Analysis of crude products was performed using analytical RP-HPLC and electrospray MS.

HPLC Purification

The crude peptide was purified by Rp-HPLC (reverse phase high performance liquid chromatography) on a Vydac C18 column, 2.5×25 cm, using a linear gradient of 10-50% acetonitrile in water, with 0.06% TFA (1%/min gradient, 10 ml/min flow rate), with monitoring by UV at 215 nm and 254 nm. Analytical HPLC was used to estimate the purity of the fractions. The final product was obtained as a lyophilized peptide with at least 95% purity estimated by analytical HPLC (Vydac C18, 0.46×25 cm, linear gradient 10-60% acetonitrile in water, 0.1% TFA, 1%/min, 1 ML/minflow rate, detection by UV absorption at 215 nm and 254 nm). The pure peptide was identified by molecular mass analysis using a SCIEX API III mass spectrometer according to standard procedures.

Analytical Data

The retention time of the peptide on RP-HPLC was 21.215 minutes. The theoretical molecular weight of the peptide was calculated to be 644.74; the actual molecular weight, through molecular mass analysis, was found to be 646.5 (MW+H*).

Coupling of the Peptide to a Carrier

Peptides were coupled to a carrier, in this case Keyhole limpet hemocyanin (KLH). Other carriers useful for such coupling include, without limitation, albumin, or ovalbumin, 8map, or lysozyme. Coupling was effected via a thioether linkage to the mercapto group of the cysteine. This type of linkage has the advantage that the peptide is coupled in a defined way to a carrier protein.

Coupling to KLH was performed as follows. 10 mg of the peptide was dissolved in 2 ml of phosphate buffered solution (PBS 1×). 1 ml of KLH (pierce products #77100) was added to the peptide solution and stirred (1 mole of peptide/50 amino acids). The KLH concentration was 10 mg/ml. 20 ul of glutaraldehyde (25% aqueous solution) was added to the peptide/carrier solution with constant stirring, incubated for 1 hour, after which a glycine stop solution was added. The peptide/carrier conjugate was separated from the peptide by dialysis against PBS

Additional YYR peptides (eg., CYYRRYYRYY SEQ ID NO:6 and CKYEDRYYRE SEQ ID NO:7) were synthesized according to standard methods, for example, those described herein. Other synthetic peptides can be prepared by making appropriate modifications of the above described synthetic methods. Such peptides are also characterized using any of the standard methods known in the art (e.g. those described herein).

Immunization of Rabbits

Polyclonal antibodies were prepared according to standard methods, and an immune response was enhanced with repeated booster injections, at intervals of 3 to 8 weeks. The success of the immunization was verified by determining the concentration of antibodies in a western blot or ELISA or both. More specifically, to generate polyclonal antibodies to misfolded PrPC (or PrPSc), the tripeptide YYR conjugated to KLH was injected into rabbits in accordance with a 164 day immunization regimen, after which the animals that had produced specific antibodies were bled.

In order to sample the serum prior to immunization, 10 ml of blood per rabbit was taken as a preimmune control. Primary immunizations were carried out with Freund's complete adjuvant and subsequent boosts with incomplete Freund's adjuvant (IFA0 (1 ml per rabbit, 0.5 ml per thigh muscle). Each injection consisted of approximately 200 ug of the purified peptide. At days 21, 42 and 70, a booster injection was given with IFA. At days 31, 42 and 80, 10 ml of blood was collected from the central ear artery for titer determination (6 ml/kg/rabbit). At day 80, the titer of the sera was checked, and 3 more injections were given (IFA) at 4 week intervals, followed by blood sampling 10 days later. 10 days after the last boost, anesthetized rabbits were exsanguinated via cardiac puncture, and antisera were collected.

Immunization of Goats

Goat polyclonal antibodies were generated according to standard methods. Three goats were immunized as follows. On day 1, all the goats received a primary immunization of 1 mg of YYR-KLH conjugates in complete Freund's adjuvant. Boosts were done by injection of 1 mg YYR-KLH in incomplete Freund's adjuvant for two of the three goats, whereas the third goat received 1 mg YYR-8map conjugates in incomplete Freund's adjuvant. Serum samples from each of the three bleeds were tested for reactivity by ELISA against YYR-BSA conjugates. From the third set of bleeds, total lgG was purified by ammonium sulfate precipitation and YYR-reactive lgG was purified using a YYR affinity column. lgG fractions were tested for reactivity to PrPSc as described herein. The exact immunization schedule was as follows: Day 1, primary immunization; D 21, first boost immunization; Day 30, first bleed; Day 46, second boost immunization; Day 53, second boost immunization; Day 60, second bleed; Day 76, third boost immunization; Day 83, third boost immunization; and Day 90, third bleed. Alternatively, monoclonal antibodies may be prepared using the synthetic peptides described herein and standard hybridoma technology (see, e.g., Kohler et al., Nature 256, 1975; Kohler et al., Eur. J. Immunol. 6:511, 1976; Kohler et al., Eur. J. Immunol. 6:292, 1976 Hammerling et al., in Monoclonal Antibodies and T Cell Hydridomas, Elsevier, N.Y., 1981; Ausubel et al., 1999, Current Protocols in Molecular Biology, Wiley Interscience, New York,) Once produced, monoclonal antibodies are also tested for specific PrP recognition by immunoprecipitation and western blot analysis (e.g., by using the methods described in Ausubel et al., supra).

Immunization of Mice

The generation of monoclonal antibodies was carried out as follows. Mice were immunized with baculovirus supernatant containing mouse PrP-AP fusion protein in complete Freund's adjuvant, then boosted 2 weeks later with the same antigen in incomplete Freund's adjuvant. Two weeks after that immunization the mice were boosted with a mixture of PrP-AP supernatant plus 100 ug of KLH-CYYRRYYRYY (SEQ ID NO:6) and 10 ug of KLH-CKYEDRYYRE (SEQ ID NO:7) conjugates. Splenocytes from these mice were fused to the FO murine B cell line (ATCC CRL-1646) to generate specific hybridoma clones. Hybridoma supernatants were screened by ELISA. There were no reactive supernatants to PrP-AP or to the CKYEDRYYRE sequence, although there were clones reactive to YYR-8map conjugates.

Purification of Antibody

Total rabbit lgG was purified from serum using the Pharmacia protein A HiTrap column according to the manufacturer's recommendations. Briefly, a HiTrap column was equilibrated with 3 column volumes of start buffer (0.2M sodium phosphate buffer, pH7.0). Serum was applied, using a syringe through a luer adaptor, onto the column. The column was subsequently washed with 5 ml of start buffer. Bound protein was eluted with 0.1M glycine, pH 3.0, and collected in eppendorf tubes containing 1M Tris ph 8.0 (50 ul/500 ul sample). Fractions were analyzed on SDA-PAGE.

Goat polyclonal antibodies were purified from serum samples as is described above.

Mouse monoclonal antibodies were produced as ascites, and purified using a protein A column kit (Pierce) according to the manufacturer's instructions. Briefly, a sample of ascites was diluted with binding buffer at a 1:1 final ratio. The sample was then added to the top of the column, which had been previously equilibrated with binding buffer, and allowed to flow through the matrix. The pass-through material was collected and the column washed with 5 volumes of binding buffer. Mild elution buffer was added to the column to release the bound lgG antibody from the matrix. Other antibody isotypes were collected by switching to the lgG elution buffer. All the antibodies were collected in 1 ml fractions, which were analyzed by BCA to determine total protein content and SDS-PAGE electrophoresis to establish the degree of antibody purity. The fraction containing the most yield of lgG was desalted by passing it through a D-salt column (Pierce). The antibody fraction was allocated and stored at −80 C. in PBS

Antibodies produced using the aforementioned procedures were subsequently tested for high-affinity binding as follows.

Immunoprecipitation

Ten ul of brain extract was added to 950 ul of Immunoprecipitation buffer (PBS 3% NP-40, 3% Tween-20) and incubated at 37 C. for 30 or 60 minutes. For experiments evaluating the reactivity of PrP 27-30 with the bead conjugates, the incubation was preceded by addition of 50 ul of 1 mg/ml proteinase K. Samples not treated with proteinase K were still incubated at 37 D. for the appropriate time period. After the incubation, 60 ul of an 100 mM PMSF solution were added to both sets of tubes. On hundred ul of resuspended bead conjugates were then added to the mixture, and incubated with rotation at room temperature for 2 hours. The beads were washed 3 times with washing buffer (PBS 2% NP-40 2% Tween-20) and resuspended by vortex after each wash. After the last wash, the beads were resuspended in 20 ul of 2× loading buffer (100 mM Tris pH 6.8, 4% SDS, 0.015% bromphenol blue, 20% glycerol) and heated at 95 C for 3 minutes.

Western Blot

The PrPSc content of brain homogenates was determined by western blotting according to standard methods. Protein samples were mixed with 2× sample buffer at a ratio of 1:1 and boiled for 5 minutes at 100 C. SDS-PAGE analysis was performed according standard methods. Samples were applied to a pre cast 15% acrylamide gels (Biorad) along with pre-stained molecular weight markers (Biorad). The gels were run at 100 V until the bromophenol blue dye front reached the bottom of the gel. The separated protein was then transferred onto PVDF membranes at 100 V or 1 hr. The membranes were washed as described above before incubation with a goat anti-mouse lgG alkaline phosphatase conjugated secondary antibody (1:5000 in TBST) for 1 hour at room temperature. After washing, signals were developed with the chemiluminescent substrate CDP-star, and exposed to x-ray films.

Flow Cytometry

Spleen cell suspensions were prepared from Balb/c mice by passing the tissues through a wire mesh. The cells were washed once with cold Dulbecco's PBS without Ca2+ or Mg2+ and viable cells were isolated by underlayering of the cell suspension with Lympholyte (Cedarlane) and centrifugation at 1300 g for 20 minutes. The cells were washed once with cold Dulbecco's PBS without CA2+ or Mg2+2.5% fetal bovine serum, and 0.5×10⁶ cells were aliquoted per well in a round bottom 96 well plate. The cells were centrifuged and resuspended in 50 ul of antibody-FITC conjugates at 1/10 final concentration in Dulbecco's PBS without CA2+ or Mg2+2.5% fetal bovine serum, for 15 minutes on ice. The cells were then washed twice with cold Dulbecco's PBS without Ca2+ or Mg2+2.5% fetal bovine serum and resuspended in the same medium containing 1 ug/ml of propidium iodide. The cells were analyzed on a Coulter Epics flow cytometer and were gated by size and granularity (forward and side scatter) and viability (exclusion of propidium iodide fluorescence).

FITC Antibody Conjugation

Fluoresceinated mAbs were made by using the Fluorotag kit (Sigma) following the manufacturer's instructions. Briefly, 0.5 mg of each antibody was raised to pH 9 with concentrated bicarbonate buffer, and FITC stock solution was added to produce an FITC: antibody ratio of 20:1. The vials were then incubated for 2 hours at room temperature. Labeled antibody was separated from free FITC by passing the mixture over a Sephadex G-25M column. Conjugated antibodies were tested for successful fluoresceination by measuring their FITC emissions at 35 nm using an LJL Biosystems Analyst, and the antibodies were tested for retention of their binding activity with an ELISA against YYR-8map conjugates.

Testing of Antibody in an ELISA

To determine whether antibody pAbC2 was useful in specifically recognizing PrPSc from bovine brain extracts, compared to PrPC using recombinant PrP (rbPrP), an ELISA approach was used. Either pools of PrPSc containing brain extracts or rbPrP was used to test the specificity of pAbC2 for PrPSc.

The wells of an Immunolon ELISA plate (Dynex) were coated overnight at 4 C. with the PC2 containing culture supernatant in a TBS buffer containing 50 mM Tris, pH 7.5, 150 mM NaCl, 1 mM CaCl2. For BSE-brain extract experiments, control wells were coated with a supernatant containing Mek-4; for rbPrP experiments, milk was used as a control to determine the non-specific binding of the antibody to the well. The coating of the ELISA plates with soluble PC2 was confirmed with an anti-PC2 monoclonal antibody. The wells were washed four times using a SLT ‘Columbus’ microplate washer (Tecan) with TBS containing 0.05% Tween 20, and blocked by filling the wells with 0.2% I-Block (Tropix) in TBST and incubating the plate at 37 C for 1 hour. The plates were washed and the bovine brain homogenate (diluted to 1% w/v in TBS) or rbPrP was added to designated wells and incubated at RT for 1 h. Wells were washed four times with TBST. pAbC2 was added to appropriate wells and incubated at RT or 1 hour, followed by a further 45 minute incubation with 100 ul of an anti-rabbit or mouse lgG/horseradish peroxidase conjugate (1:5000) in TBST containing 1% nonfat milk. Wells were washed four times with TBST. Signals were developed with TMB/H2O2 as a substrate for peroxidase. Reactions were stopped after 15 minutes by the addition of 100 ul of 2M phosphoric acid. Signals were monitored at 450 nm with reference at 620 nm using a SLT microplate reader. Specific positive signals were determined by comparing PrP binding to PC2 with PrP binding to the negative control, Mek-4 or milk. Preimmune controls showed no binding.

It will thus be appreciated that various antibodies that recognize and bind selectively to the YYR epitope unique to misfolded PrPC (shown herein above using an aggregated form known as PrPSc) can be raised and used in accordance standard practices well established for these purposes.

### Example 2

### Cancer Cell Screening

In a series of experiments using tumor cells stained with the Tyr-Tyr-Arg IgG mouse monoclonal antibody 4C2 at 5-10 micrograms per mL and analyzed by flow cytometry as described above, it was found that a proportion of tumor cells (10-50%) possess cell surface immunoreactivity for this misfolding-specific epitope. These tumor cell lines were from lymphoid (MOLT-4), myeloid (HL-60) and oligodendroglial lineages (MO3.13), which also possessed cell surface PrPC as defined by the monoclonal antibodies 3F4 and 6H4. 4C2 immunoreactivity could not cloned to 100% by limiting dilution of tumor cells, suggesting that all such cells pass through a phase of growth in which Tyr-Tyr-Arg is exposed at the cell surface. It was also found that surface 4C2 immunoreactivity on these tumor cell lines was not as sensitive as 3F4 or 6H4 immunoreactivity to hydrolysis by phosphatidylinositol-specific phospholipase C (PIPLC), or by proteolysis by trypsin. Moreover, it was also found that deliberate denaturation of mouse Prnp null dissociated brain cells by brief exposure to low pH also evoked cell surface immunoreactivity for Tyr-Tyr-Arg. These data indicate that some tumor cells possess Tyr-Tyr-Arg immunoreactive misfolded proteins at the cell surface, including but not limited to the prion protein.

The experimental protocols used to identify cancer cells presenting a misfolded surface protein were as follows. The same protocols are useful to identify other cancer cells presenting with a misfolded surface protein.

Cells

Human lymphoid (MOLT-4), myeloid (HL60), and neural (MO 3.13) cells were maintained in suspension and in monolayer respectively in vitro. A penicillin/streptomycin cocktail was used to prevent bacterial growth. Cells were split approximately once a week with a change of media 2 to 3 days after splitting, they were collected on the mornings of experiments. Cells were centrifuged at 500 g for 10 minutes and then subjected to various treatments and labeling with antibodies.

Low pH Treatment

Phosphate buffered saline (PBS) adjusted to pH 3.5 was used to treat cells in order to misfold the cell surface proteins, including the prion protein. Cells were incubated with pH 3.5 (15 mL of PBS to approximately 60×10⁶ cells) PBS for 15 minutes at room temperature. They were then washed twice with PBS+10% normal goat serum (NGS) and labeled with appropriate antibodies.

Trypsin Treatment

In experiments that studied the effect of the proteolytic enzyme trypsin on a the cell surface proteins of a population of cells, the cells were collected from a culture flask the morning of the experiment and centrifuged at 500 g for 10 minutes. The cells were then treated with 0.5% trypsin (15 mL of trypsin to approximately 60×10⁶ cells) for 30 minutes at room temperature. They were then washed twice with PBS+10% NGS and labeled with appropriate antibodies. DNase (0.03 mg/mL) was used to obviate cell clumping.

PI-PLC Treatment

Phosphatidylinositol-specific phospholipase C (PI-PLC) (Molecular Probes Inc., Eugene, Oreg.) is an enzyme which hydrolyzes glycosylphosphatidyl inositol anchors of surface proteins. In removing GPI-anchored proteins from cell surfaces 1 unit/mL of PI-PLC was used in cells suspended in 100 μL of PBS+10% NGS. They were then rotated for one hour at 37° C. The cells were then washed twice with PBS+10% NGS and labeled with appropriate antibodies.

Antibody Labeling

Cells treated or untreated with low pH, trypsin or PI-PLC were washed twice and incubated for 30 minutes on ice with an appropriate primary antibody (used at 5-10 micrograms/mL). The cells were then washed twice with PBS+1% NGS and resuspended in 100 μL of PBS+10% NGS. The appropriate secondary antibody was then added and the cells were again incubated on ice for 30 minutes. After again being washed twice and resuspended in 100 μL of PBS+10% NGS, 7AAD (used as a fluorescent vital dye) was added. The cells were then suspended in 1 mL of PBS ready for flow cytometry.

### Primary Antibodies.

3F4 (Signet)—Human anti-PrP antibody
6H4 (Prionics)—Mouse/Human anti-PrP antibody
4C2 (Cashman Lab)—Human anti-PrP^{Sc} (YYR) antibody

### Secondary Antibodies

Goat Anti-mouse FITC (BD Biosciences)
Streptavidin-PE
   7AAD (BD Biosciences)—DNA binding dye (to exclude dead cells from flow cytometry analysis)

### Flow Cytometry

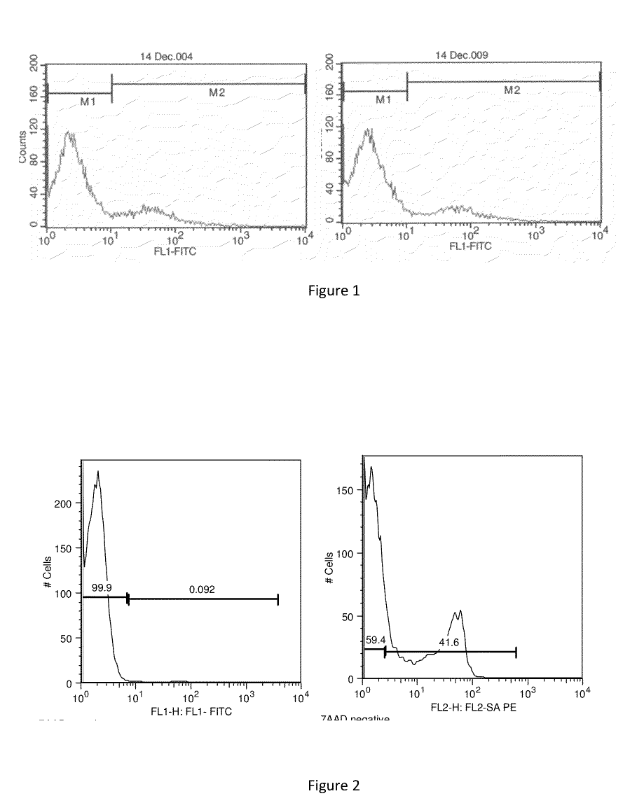

The Becton Dickinson FACSCalibur (BD Biosciences) flow cytometer was used to perform fluorescence analysis of labeled cells. Dot plots and histograms were collected of Side Scatter (SSC), Forward Scatter (FSC), fluorescent channel one (FL1-FITC), fluorescent channel two (FL2-PE) and fluorescent channel three (FL3-7AAD). Fluorescence intensity less than 10¹ is considered negative and above is considered positive on both dot plots and histograms. Results are shown in FIGS. 1 and 2.

It will thus be appreciated that cancer cells can be screened to detect on their surface a misfolded form of PrPC, or a misfolded form of a protein other than PrPC. In embodiments of the present invention, this screening process is applied to cancer cells extracted from a subject presenting with a cancer, such as a leukemia, a lymphoma or another type of hematopoietic or solid tumour. The cells are screened using the method detailed above. Subjects testing positive for the presence of misfolded surface protein are thus identified as candidates for therapy, using the antibodies or conjugated herein described, which bind to the detected misfolded protein and reduce the viability of the targeted cells in the subject, as evidenced by an inhibition in the growth or proliferation of those cells. Such inhibition can be determined, and clinical regression of the disease can be monitored, using the misfolded protein screening protocols just described, with a therapeutic benefit being revealed as a reduction in the number, size, metabolism or location of misfolded protein positive cells.

I claim:

1. A method for treating a subject to inhibit the growth or proliferation of a cancer cell presented by said subject, comprising the step of administering to the subject an effective amount of an agent that is a) an antibody that binds selectively to an epitope unique to a misfolded form of a surface protein presented by the cancer cell or b) a vaccine effective in said subject to elicit the production of said antibody, wherein said antibody binds to an epitope unique to a misfolded form of prion protein PrP, presented by the cancer cell.

2. The method according to claim 1, wherein the epitope unique to the misfolded form of PrP is a YYX epitope.

3. The method according to claim 2, wherein said YYX epitope is the YYR epitope.

4. The method according to claim 3, wherein the agent comprises an anti-YYR antibody or a YYR binding fragment thereof.

5. The method according to claim 4, wherein the agent comprises an anti-YYR antibody.

6. The method according to claim 5, wherein said cancer cell is selected from a hematopoietic cancer cell or a solid tumor cell.

7. The method according to claim 6, wherein the cancer cell is selected from carcinoma, lymphoma, blastoma, sarcoma, and leukemia.

8. The method according to claim 7, wherein the cancer cell is a constituent of cancer selected from the group consisting of cancer of the breast, prostate, colon, lung, squamous tissue, gastrointestinal tract, pancreas, brain, cervix, ovary, vulva, liver, bladder, kidney, colon, salivary gland, thyroid gland, and head and neck.

9. The method according to claim 1, wherein the agent administered to said subject is said vaccine effective in said subject to elicit production of said antibody.

10. The method according to any claim 9, wherein said vaccine is effective to elicit production of said antibody.

11. A conjugate comprising a cytotoxin and an agent that binds selectively to an epitope presented by an epitope unique to the misfolded form of a cancer cell surface protein.

12. The conjugate according to claim 11, wherein the agent binds selectively to a YYX epitope.

13. The conjugate according to claim 12, wherein the agent that binds a YYX epitope is an anti-YYX antibody.

14. The conjugate according to claims 13, wherein the cytotoxin is selected from a chemotherapeutic agent, a toxin, and a radioisotope.

15. A conjugate according to any of claims 14, wherein said YYX epitope is a YYR epitope.

16. A pharmaceutical composition comprising a conjugate according to claim 15, and a physiologically tolerable vehicle therefor.

17. A method for screening cancer cells, comprising the step of obtaining a sample comprising cancer cells, and assaying said cancer cells for the presence of an epitope unique to a misfolded form of a cancer cell surface protein.

18. The method according to claim 17, comprising the further step of treating said subject with an effective amount of an agent that binds selectively to the unique epitope to inhibit growth or proliferation of cancer cells positive for said epitope.

19. The method according to claim 18, wherein said epitope is the YYR epitope.


SEQUENCE LISTING (7 sequences)

SEQ ID NO: 1 - protein, 7 aa, Homo sapiens
  MISC_FEATURE (1)..(7): Each occurrence of X may, independently, be any amino acid or maybe absent.  Preferably, at least one occurrence of X, if present, is not Y.
XYYXYYX

SEQ ID NO: 2 - protein, 13 aa, Homo sapiens
  MISC_FEATURE (1)..(13): Each occurrence of X may, independently, be any amino acid or maybe absent.  Preferably, at least one occurrence of X, if present, is not Y.
XYYXXYYXYYYYX

SEQ ID NO: 3 - protein, 10 aa, Homo sapiens
  MISC_FEATURE (1)..(1): X may be any amino acid or may be absent.
XYYRRYYRYY

SEQ ID NO: 4 - protein, 9 aa, Homo sapiens
YYRRYYRYY

SEQ ID NO: 5 - protein, 4 aa, Homo sapiens
RYYC

SEQ ID NO: 6 - protein, 10 aa, Homo sapiens
CYYRRYYRYY

SEQ ID NO: 7 - protein, 10 aa, Homo sapiens
CKYEDRYYRE